(19) United States
(12) Patent Application Publication
(10) Pub. No.: US 2024/0390082 A1
(43) Pub. Date: Nov. 28, 2024

(54) CONTINUUM ROBOT CONTROL SYSTEM, CONTINUUM ROBOT CONTROL METHOD, AND RECORDING MEDIUM

(71) Applicant: CANON KABUSHIKI KAISHA, Tokyo (JP)

(72) Inventor: HIDEKAZU KOSE, Tokyo (JP)

(21) Appl. No.: 18/790,591

(22) Filed: Jul. 31, 2024

Related U.S. Application Data
(63) Continuation of application No. PCT/JP2023/001646, filed on Jan. 20, 2023.

(30) Foreign Application Priority Data
Feb. 3, 2022 (JP) 2022-015558

(51) Int. Cl. A61B 34/30; A61B 1/00; A61B 17/00; A61B 17/34; A61B 90/00; A61M 25/01

(52) U.S. Cl. CPC A61B 34/30; A61B 1/00154; A61B 17/3423; A61B 2017/00327; A61B 2034/301; A61B 2090/067; A61M 25/0147; A61M 2025/0166

(57) ABSTRACT

A control apparatus, in a case where a characteristic region related to a path of a lumen of a subject greater than or equal to a predetermined area is included in a field of view of an imaging unit in bending a bending section of a continuum robot in a predetermined direction, estimates an angle restriction value for a bending angle of the bending section based on an end position of the bending section detected after insertion of the bending section into the lumen and structural information about the lumen. In bending the bending section in the predetermined direction, the control apparatus restricts driving of a driving unit so that the bending section bends within the range of the estimated angle restriction value.

### CROSS-REFERENCE TO RELATED APPLICATIONS

This application is a Continuation of International Patent Application No. PCT/JP2023/001646, filed Jan. 20, 2023, which claims the benefit of Japanese Patent Application No. 2022-015558, filed Feb. 3, 2022, both of which are hereby incorporated by reference herein in their entireties.

### BACKGROUND OF THE INVENTION

### Field of the Invention

The present invention relates to a continuum robot control system and a continuum robot control method for controlling a continuum robot including an imaging unit, and a recording medium.

### Background Art

Minimally invasive medicine for reducing burden on an examinee, such as a patient, and improving the quality of life (QOL) after treatment or examination has been attracting attention in recent years. Representative examples of minimally invasive medicine include surgery and examination using an endoscope. For example, laparoscopic surgery enables surgical incisions of reduced size compared to conventional open surgery, and can thus not only shorten the post-operative hospital stay but also provide cosmetic benefits.

Flexible endoscopes are known as endoscopes used in minimally invasive medicine. Flexible endoscopes have an insertion portion to be inserted into the examinee's body, made of bendable members. The flexible endoscope can thus be inserted into the examinee's body without compressing tissues, even in curved organs such as the esophagus, colon, and lung, whereby the burden on the examinee can be reduced. The burden on the examinee is expected to be further reduced by driving the insertion portion of the flexible endoscope using an actuator and automatically controlling the orientation of the insertion portion to follow the path inside the examinee's body. The mechanisms and control methods of continuum robots that can be used as flexible endoscopes have therefore been under active research and development.

When inserting such a continuum robot into the lumen of a subject, the user, such as a doctor, desirably operates the continuum robot in a way that avoids strong contact with the lumen. The reason is that if the continuum robot contacts the lumen, the force acting between the continuum robot and the lumen can move the continuum robot in a direction different from the user-intended direction and thereby deteriorate the operability. Moreover, a strong contact between the continuum robot and the lumen may damage the continuum robot. According to the conventional art, the user operates the continuum robot while referring to two-dimensional images such as images from an imaging unit (camera) installed on the insertion portion of the continuum robot and medical images generated by preoperative computed tomography (CT) or magnetic resonance imaging (MRI) scans. However, since the insertion portion of the continuum robot inside the examinee's body is unable to be directly observed, the user's being proficient in manipulation techniques may be desirable to avoid accidental operation of the continuum robot in a direction of causing strong contact with the lumen.

To address such issues, PTL 1 discusses an example of a continuum robot that restricts the bending angle of its bending section, or insertion portion, based on the volume of the organ to be examined or treated. Specifically, according to PTL 1, with the heart as the target organ, a working space having a volume equivalent to that of the heart is defined, and the bending angle of the bending section, or insertion portion, is controlled so that the operating range of the distal end of the continuum robot is restricted within the working space. This can reduce the risk of the continuum robot making strong contact with the organ due to the user's operational mistakes.

### CITATION LIST

### Patent Literature

PTL 1: Japanese Unexamined Patent Application Publication No. 2007-527296

However, regarding the technique discussed in the foregoing PTL 1, the working space to restrict the bending angle of the bending section, or insertion portion, of the continuum robot can be difficult to define based on the volume of the organ, depending on the organ to be examined or treated and the procedure. Examples of such a procedure include lung biopsy for sampling tissues suspected to be lesions from the deep parts of the lung. Specifically, in the lung biopsy, the user, such as a doctor, initially inserts the continuum robot into the trachea through the examinee's mouth or nose. The user then operates the continuum robot to follow the shape of the examinee's bronchi while referring to image information from the imaging unit (camera) located at the end of the continuum robot. To apply the technique discussed in the foregoing PTL 1 to the lung biopsy, defining the working space to accurately match the shape of the examinee's bronchi is desirable. The reason is that the bronchi have a diameter only slightly greater than that of the continuum robot. Even a slight deviation between the working space of the continuum robot defined before operation and the actual shape of the bronchi can thus allow bending that should be restricted. Furthermore, the working space for the lung is difficult to accurately define. This is because the bronchi have complex three-dimensional curvatures, and the shape of the bronchi even changes with the examinee's breathing. For subjects such as the lung, it is therefore desirable to define the working space of the continuum robot using a method different from that discussed in PTL 1, so that the risk of operating the continuum robot in a direction of causing strong contact with the lumen of the subject can be reduced.

### SUMMARY OF THE INVENTION

The present invention is directed to providing a mechanism capable of reducing the risk of operating a continuum robot in a direction of causing strong contact with a lumen of a subject.

According to an aspect of the present invention, a continuum robot control system includes a continuum robot including a bending section configured to be bent with respect to a reference axis by a linear member being driven, a driving unit configured to drive the linear member, and an imaging unit located near an end of the bending section, and a control apparatus configured to control operation of the continuum robot. The control apparatus includes an angle estimation unit configured to, in a case where a characteristic region related to a path of a lumen of a subject greater than or equal to a predetermined area is included in a field of view of the imaging unit in bending the bending section in a predetermined direction, estimate an angle restriction value for a bending angle of the bending section based on an end position of the bending section detected after insertion of the bending section into the lumen and structural information about the lumen, and an angle restriction unit configured to, in bending the bending section in the predetermined direction, restrict driving of the driving unit so that the bending section bends within a range of the angle restriction value.

The present invention also includes a continuum robot control method for the foregoing continuum robot control system.

Further features of the present invention will become apparent from the following description of exemplary embodiments with reference to the attached drawings.

### BRIEF DESCRIPTION OF THE DRAWINGS

FIG. 1 is a schematic diagram illustrating an example of a general configuration of a continuum robot control system according to a first exemplary embodiment of the present invention.

FIG. 2 is a schematic diagram illustrating an example of a general configuration of a continuum robot according to the first exemplary embodiment of the present invention.

FIG. 3 is a schematic diagram illustrating an example of a general configuration of a bending section illustrated in FIG. 2.

FIG. 4 is a schematic diagram illustrating a robot coordinate system and a camera coordinate system used for control by the continuum robot control system according to the first exemplary embodiment of the present invention.

FIG. 5 is a schematic diagram illustrating an example of a general configuration of a control apparatus according to the first exemplary embodiment of the present invention.

FIG. 6 is a flowchart illustrating an example of a processing procedure for iterative calculation by which an angle restriction value estimation unit of FIG. 5 determines a bending angle restriction value.

FIG. 7 is a schematic diagram illustrating an example of a functional configuration of the angle restriction value estimation unit that performs the processing of step S103 in FIG. 6.

FIG. 8A is a diagram illustrating the first exemplary embodiment of the present invention, illustrating an example of an orientation of the continuum robot inside a subject.

FIG. 8B is a diagram illustrating the first exemplary embodiment of the present invention, illustrating another example of the orientation of the continuum robot inside the subject.

FIG. 8C is a diagram illustrating the first exemplary embodiment of the present invention, illustrating still another example of the orientation of the continuum robot inside the subject.

FIG. 9A is a diagram illustrating the first exemplary embodiment of the present invention, illustrating an example of a camera image output by an imaging unit when the continuum robot assumes the orientation of FIG. 8A.

FIG. 9B is a diagram illustrating the first exemplary embodiment of the present invention, illustrating an example of the camera image output by the imaging unit when the continuum robot assumes the orientation of FIG. 8B.

FIG. 9C is a diagram illustrating the first exemplary embodiment of the present invention, illustrating an example of the camera image output by the imaging unit when the continuum robot assumes the orientation of FIG. 8C.

FIGS. 10A to 10C are diagrams illustrating an example of a mode where the bending angle restriction value for the bending section of the continuum robot according to the first exemplary embodiment of the present invention can be increased.

FIG. 11 is a schematic diagram illustrating an example of a general configuration of a continuum robot control system according to a second exemplary embodiment of the present invention.

FIG. 12 is a schematic diagram illustrating an example of a general configuration of a control apparatus according to the second exemplary embodiment of the present invention.

FIG. 13A is a diagram illustrating the second exemplary embodiment of the present invention, illustrating an example of the orientation of the continuum robot inside a subject.

FIG. 13B is a diagram illustrating the second exemplary embodiment of the present invention, illustrating another example of the orientation of the continuum robot inside the subject.

FIG. 13C is a diagram illustrating the second exemplary embodiment of the present invention, illustrating still another example of the orientation of the continuum robot inside the subject.

FIG. 14A is a diagram illustrating the second exemplary embodiment of the present invention, illustrating an example of a camera image output by an imaging unit when the continuum robot assumes the orientation of FIG. 13A.

FIG. 14B is a diagram illustrating the second exemplary embodiment of the present invention, illustrating an example of the camera image output by the imaging unit when the continuum robot assumes the orientation of FIG. 13B.

FIG. 14C is a diagram illustrating the second exemplary embodiment of the present invention, illustrating an example of the camera image output by the imaging unit when the continuum robot assumes the orientation of FIG. 13C.

FIG. 15 is a schematic diagram illustrating an example of a general configuration of a continuum robot control system according to a third exemplary embodiment of the present invention.

FIG. 16 is a schematic diagram illustrating an example of a plurality of bending sections included in a continuum robot according to the third exemplary embodiment of the present invention.

FIG. 17 is a schematic diagram illustrating an example of a general configuration of a control apparatus according to the third exemplary embodiment of the present invention.

### DESCRIPTION OF THE EMBODIMENTS

Modes (exemplary embodiments) for carrying out the present invention will be described below with reference to the drawings.

A first exemplary embodiment of the present invention will initially be described.

In the present exemplary embodiment, an example of a continuum robot control system including a continuum robot that includes a three-dimensionally bendable bending section and a control apparatus that controls operation of the continuum robot will be described. A configuration of the continuum robot control system according to the present exemplary embodiment will initially be described. Next, a configuration of the continuum robot according to the present exemplary embodiment will be described. A method by which the control apparatus restricts the bending angle of the bending section will then be described, and finally an example of a procedure for sampling a specimen from a deep part of the lung (subject) of a patient or another examinee will be explained.

### [1-1: Configuration of Continuum Robot Control System]

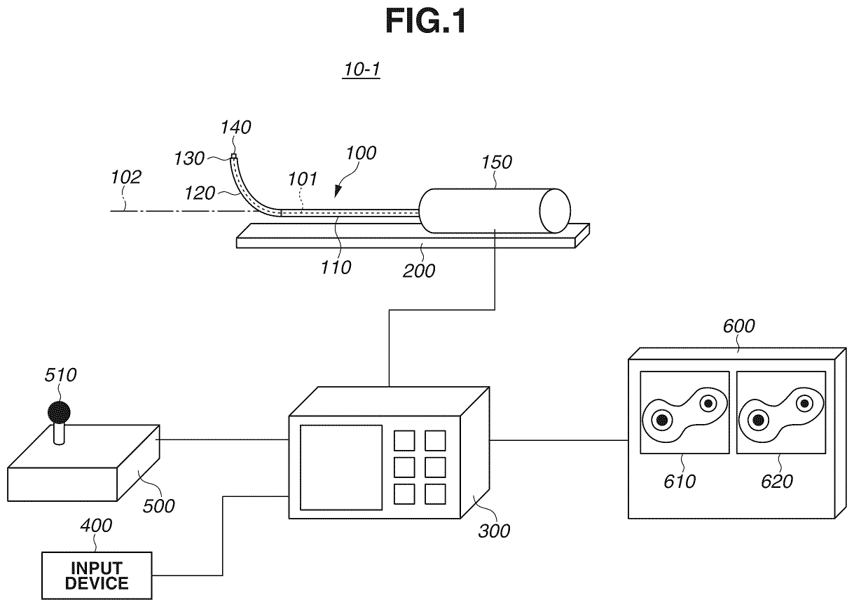

FIG. 1 is a schematic diagram illustrating an example of a general configuration of a continuum robot control system 10-1 according to the first exemplary embodiment of the present invention. As illustrated in FIG. 1, the continuum robot control system 10-1 includes a continuum robot 100, a linear stage 200, a control apparatus 300, an input device 400, an operation device 500, and an image display device 600.

As illustrated in FIG. 1, the continuum robot 100 includes an elongated section 110, a bending section 120, a coil 130, an imaging unit 140, and a driving unit 150. The continuum robot 100 also includes a tool channel 101 that is a tube-like passage running through the elongated section 110 and the bending section 120. The tool channel 101 is intended to insert and remove various tools through a tool insertion port located near the junction between the elongated section 110 and the driving unit 150. Examples of the various tools to be inserted and removed into/from the tool channel 101 include an imaging tool with the imaging unit 140 at its tip, and surgical tools such as a biopsy brush tool, a biopsy needle tool, and other biopsy tools.

In addition to the tool channel 101, the elongated section 110 also includes a plurality of drive wires passed therethrough. The plurality of drive wires corresponds to a plurality of linear members to be driven by the driving unit 150 in bending the bending section 120 with respect to a reference axis 102.

The bending section 120 is configured to be capable of actively changing orientation. Specifically, the bending section 120 bends with respect to the reference axis 102 when the drive wires, or the linear members connected to the bending section 120, are driven by actuators (driving sections) disposed in the driving unit 150. In the present exemplary embodiment, the reference axis 102 is an axis in a direction parallel to the moving direction of the continuum robot 100 on the linear stage 200.

The coil 130 is disposed at the end of the bending section 120. Although not illustrated in FIG. 1, a magnetic field generator is located near the bending section 120. The control apparatus 300 can detect the end position and direction of the bending section 120 by reading a change in the magnetic field generated by the magnetic field generator (not illustrated) via the coil 130.

The imaging unit 140 is a component having a camera function, disposed at the end of the imaging tool inserted into the tool channel 101, for example. The tool channel 101 is equipped with guide members, for example, and the imaging unit 140 of the imaging tool inserted into the tool channel 101 is located at a predetermined insertion depth and in a predetermined phase near the end of the bending section 120.

The driving unit 150 includes the actuators (driving sections) for driving the drive wires, or the linear members connected to the bending section 120, when bending the bending section 120 at a predetermined bending angle with respect to the reference axis 102. In the present exemplary embodiment, the driving unit 150 is fixed to the linear stage 200. The continuum robot 100 is linearly moved in the longitudinal direction of the linear stage 200 by the user, such as a doctor, pushing and pulling the driving unit 150 forward and backward.

As described above, the driving unit 150 is fixed to the linear stage 200. The linear stage 200 corresponds to a moving device that moves the continuum robot 100 forward and backward with respect to the examinee (subject).

The control apparatus 300 is an apparatus that controls operation of the continuum robot 100 based on an operation input from the operation device 500, input from the input device 400, input from the coil 130, and an image output from the imaging unit 140, for example. The control apparatus 300 also performs various types of control including a display control of the image display device 600, and various types of processing.

The input device 400 is a device for inputting various types of information (including various types of data and various images) to the control apparatus 300.

The operation device 500 is a device for the user, such as a doctor, to operate. This operation device 500 includes a lever 510 for the user, such as a doctor, to operate so that the bending section 120 assumes a desired orientation. The control apparatus 300 outputs wire driving commands to the actuators (driving sections) of the driving unit 150 so that the bending section 120 assumes the desired orientation, based on the amount of operation of the lever 510.

The control apparatus 300 includes an interface for receiving an image obtained by the imaging unit 140. The image received from the imaging unit 140 by the control apparatus 300 is output to the image display device 600 and displayed as a camera image 610. In addition to the camera image 610 output from the imaging unit 140, the image display device 600 displays a navigation image 620 that is generated from a three-dimensional (3D) model of the examinee's lung constructed before operation, for example. Examples of the navigation image 620 include an image observing the interior of the lumen in the 3D model of the lung that is the subject from a first-person point of view, and a bird's-eye view observing the 3D model of the lung that is the subject from outside the subject. The user, such as a doctor, can switch the images as appropriate.

### [1-2: Configuration of Continuum Robot and Coordinate Systems]

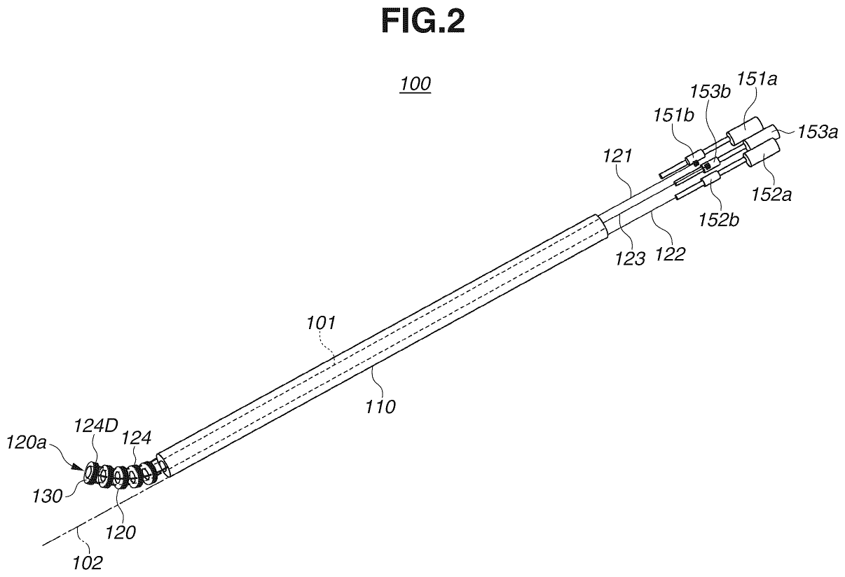

FIG. 2 is a schematic diagram illustrating an example of a general configuration of the continuum robot 100 according to the first exemplary embodiment of the present invention. In FIG. 2, components similar to those illustrated in FIG. 1 are denoted by the same reference numerals. A detailed description thereof will be omitted. FIG. 2 does not include the imaging unit 140 illustrated in FIG. 1.

The elongated section 110 is a member to be passively bent by external force.

The bending section 120 includes a plurality of drive wires 121 to 123 that is a plurality of linear members, and a plurality of wire guides 124 that are members for guiding the plurality of drive wires 121 to 123. Here, three drive wires 121 to 123 are fixedly connected at one end to a wire guide 124D located at an end 120a of the bending section 120, and are connected at the other end to actuators 151a to 153a via drive transmission mechanisms. For example, the wire guide 124D located at an end 120a of the bending section 120 is equipped with the foregoing coil 130.

The driving unit 150 illustrated in FIG. 1 includes the actuators 151a to 153a and feed screws 151b to 153b illustrated in FIG. 2. Specifically, the drive wire 121 is connected to the actuator 151a via the feed screw 151b. The drive wire 122 is connected to the actuator 152a via the feed screw 152b. The drive wire 123 is connected to the actuator 153a via the feed screw 153b. The actuators 151a to 153a are driven to push or pull the respective drive wires 121 to 123 in the longitudinal direction of the continuum robot 100 based on control of the control apparatus 300, whereby the bending section 120 can be bent with respect to the reference axis 102.

The behavior of the bending section 120 and the elongated section 110 in driving the actuators 151a to 153a will now be described.

The rotational motions of the actuators 151a to 153a are decelerated by the feed screws 151b to 153b connected to the respective output axes and converted into translational motions. The nuts of the feed screws 151b to 153b are equipped with wire gripping units for fixing the drive wires 121 to 123. Driving the actuators 151a to 153a pushes or pulls the drive wires 121 to 123 in the longitudinal direction of the continuum robot 100. Here, the drive wires 121 to 123 are fixedly connected to the wire guide 124D at the end 120a of the bending section 120 with respective different phases. The bending section 120 can thus be bent to a desired bending angle and direction by controlling the driving amounts of the respective actuators 151a to 153a (pushing or pulling amounts of the respective drive wires 121 to 123). Since the drive wires 121 to 123 are not fixed to the elongated section 110, the orientation of the elongated section 110 remains unchanged when the drive wires 121 to 123 are pushed or pulled.

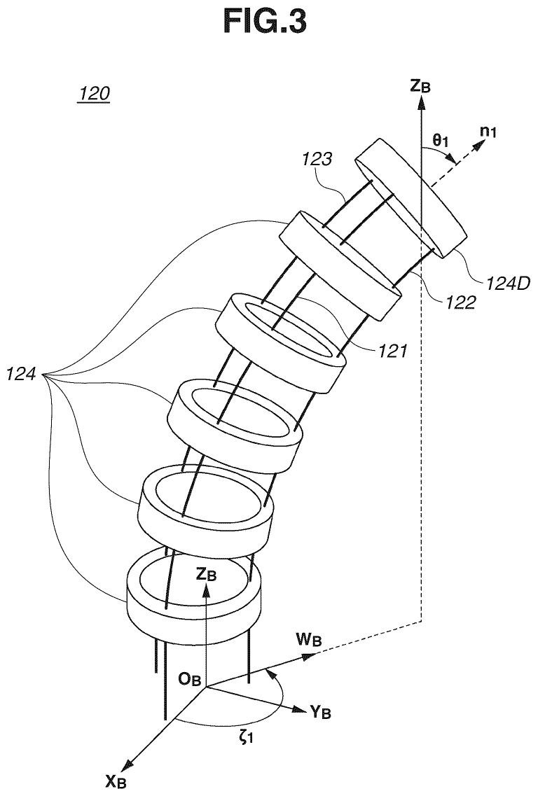

FIG. 3 is a schematic diagram illustrating an example of a general configuration of the bending section 120 illustrated in FIG. 2. In FIG. 3, components similar to those illustrated in FIG. 2 are denoted by the same reference numerals. A description thereof will be omitted.

The three drive wires 121 to 123 are all connected to the wire guide 124D located at the end (hereinafter, may be referred to as a “distal end” as appropriate) 120a of the bending section 120. By contrast, only the drive wire 121 is connected to the wire guides 124 other than the wire guide 124D. The drive wires 122 and 123 can slide through not-illustrated guide holes formed in the wire guides 124 in the longitudinal direction of the continuum robot 100.

Next, the kinematics of the continuum robot 100 will be derived by introducing coordinate systems to be used for control by the continuum robot control system 10-1 according to the exemplary embodiment and variables indicating the orientation of the bending section 120.

The control by the continuum robot control system 10-1 according to the present exemplary embodiment uses a working coordinate system with reference to the examinee, such as a patient, a robot coordinate system with reference to the driving unit 150, and a camera coordinate system with reference to the end 120a of the bending section 120. In the working coordinate system, a predetermined position in the trachea of the examinee is assumed as an origin O_{W}, the direction from the larynx to the lung of the examinee as a Z-axis Z_{W}, and the direction from the abdomen to the back as a Y-axis Y_{W}. An X-axis X_{W} is assumed to form a right-handed coordinate system with the Y- and Z-axes Y_{W} and Z_{W}.

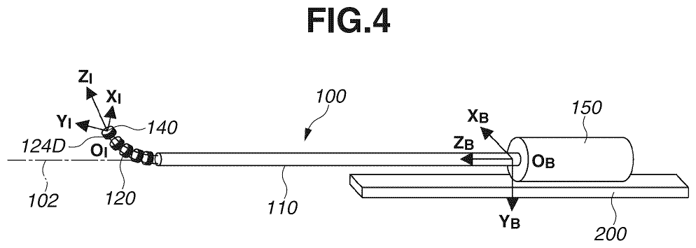

FIG. 4 is a schematic diagram illustrating the robot coordinate system and the camera coordinate system to be used for the control by the continuum robot control system 10-1 according to the first exemplary embodiment of the present invention. In FIG. 4, components similar to those illustrated in FIGS. 1 to 3 are denoted by the same reference numerals. A detailed description thereof will be omitted. In the following description, the drive wire 121 illustrated in FIGS. 2 and 3 will be referred to as a “1a-wire”, the drive wire 122 illustrated in FIGS. 2 and 3 as a “1b-wire”, and the drive wire 123 illustrated in FIGS. 2 and 3 as a “1c-wire”.

In the robot coordinate system, as illustrated in FIG. 4, the center of the base portion of the continuum robot 100 near the junction between the elongated section 110 and the driving unit 150 is assumed as an origin OB. In the robot coordinate system, as illustrated in FIG. 4, the longitudinal direction of the elongated section 110 is assumed as a Z-axis Z_{B}, and the direction of the 1a-wire with reference to the origin OB as an X-axis X_{B}. A Y-axis Y_{B} is assumed to form a right-handed coordinate system with the X- and Z-axes X_{B} and Z_{B}. As illustrated in FIG. 4, the Z-axis Z_{B} is equivalent to the reference axis 102.

The position and orientation of the imaging unit 140 (camera) with respect to the end (distal end) 120a of the bending section 120 are determined by the guide members disposed in the foregoing tool channel 101. In the present exemplary embodiment, the camera coordinate system is defined with reference to the distal end of the continuum robot 100. The foregoing guide members define the insertion amount of the imaging unit 140 so that the center of a light receiving portion of the imaging unit 140 matches the center of the wire guide 124D at the distal end 120a of the bending section 120. As illustrated in FIG. 4, the center of the wire guide 124D is thus assumed as an origin Or of the camera coordinate system. The foregoing guide members also define the phase of the imaging unit 140 so that the direction from the origin O₁ illustrated in FIG. 4 to the 1a-wire matches the X-axis of the camera image. This direction is assumed as an X-axis X_{I} of the camera coordinate system. In the camera coordinate system, as illustrated in FIG. 4, the line of sight direction of the imaging unit 140 is assumed as a Z-axis Z_{I}. A Y-axis Y_{I} is assumed to form a right-handed coordinate system with the X- and Z-axes X_{I} and Z_{I}.

In the following description, when a vector is used, a top left superscript will be used to indicate which of the foregoing coordinate systems the vector is defined in. Specifically, the top left superscript W indicates that the vector is observed in the working coordinate system, the top left superscript B in the robot coordinate system, and the top left superscript I in the camera coordinate system. For example, the end position vectors of the bending section 120 expressed in the robot coordinate system and the working coordinate system are ^{B}p₁ and ^{W}p₁, respectively.

As variables indicating the orientation of the bending section 120, a bending angle θ₁ indicating the magnitude of bending and a turning angle ζ₁ indicating the turning direction are defined as illustrated in FIG. 3. Specifically, as illustrated in FIG. 3, the bending angle θ₁ of the bending section 120 refers to an angle formed between a longitudinal unit vector n₁ at the distal end 120a of the bending section 120 and the Z-axis Z_{B} of the robot coordinate system (as illustrated in FIG. 4, may be regarded as the “reference axis 102”). As illustrated in FIG. 3, the turning angle ζ₁ of the bending section 120 refers to an angle formed between a vector WB and the X-axis X_{B}. The vector WB is the unit vector n₁ projected on the X_{B}-Y_{B} plane.

The kinematics (hereinafter, referred to as “actuator kinematics”) representing the relationship between the bending angle θ₁ and the turning angle ζ₁ of the bending section 120 and the driving amounts 1₁ₐ, 1_{1b}, and 1_{1c} of the 1a-, 1b-, and 1c-wires is expressed by the following Eqs. (1) to (3), respectively:

$${l}_{1 o} = \frac{{R}_{g} {θ}_{1}}{2} cos ( {ξ}_{1} - {ζ}_{1} ) \qquad ( 1 )$$

$${l}_{1 b} = \frac{{R}_{g} {θ}_{1}}{2} cos ( {ξ}_{1} - {ζ}_{1} + \frac{2 π}{3} ) \qquad ( 2 )$$

$${l}_{1 c} = \frac{{R}_{g} {θ}_{1}}{2} cos ( {ξ}_{1} - {ζ}_{1} + \frac{4 π}{3} ) \qquad ( 3 )$$

The kinematics (hereinafter, referred to as “robot kinematics”) representing the relationship between a position p₁ and direction n₁ of the end 120a of the bending section 120 in the robot coordinate system and the bending angle θ₁ and turning angle ζ₁ of the bending section 120 is expressed by the following Eqs. (4) and (5), respectively:

$${}^{B} {p}_{1} = [ \begin{matrix}\frac{\text{?}}{{θ}_{1}} ( 1 - cos {θ}_{1} ) cos {ζ}_{1} & \frac{\text{?}}{{θ}_{1}} ( 1 - cos {θ}_{1} ) sin {ζ}_{1} & \frac{\text{?}}{{θ}_{1}} sin {θ}_{1}\end{matrix} ] \qquad ( 4 )$$

$$\text{?} = [ \begin{matrix}sin {θ}_{1} cos {ζ}_{1} & sin {θ}_{1} sin {ζ}_{1} & cos {θ}_{1}\end{matrix} ] \qquad ( 5 )$$

$$\text{?} \text{indicates text missing or illegible when filed}$$

In Eqs. (1) to (5), R_{g} represents the diameter of a pitch circle of the wires passing through the wire guides 124, and 1_{1d} represents the length of the center axis of the bending section 120. Si represents the phase of the guide holes in the wire guides 124 with respect to the Z_{B} axis of the robot coordinate system. For the bending section 120, ζ1=0.

### [1-3: Configuration of Control Apparatus]

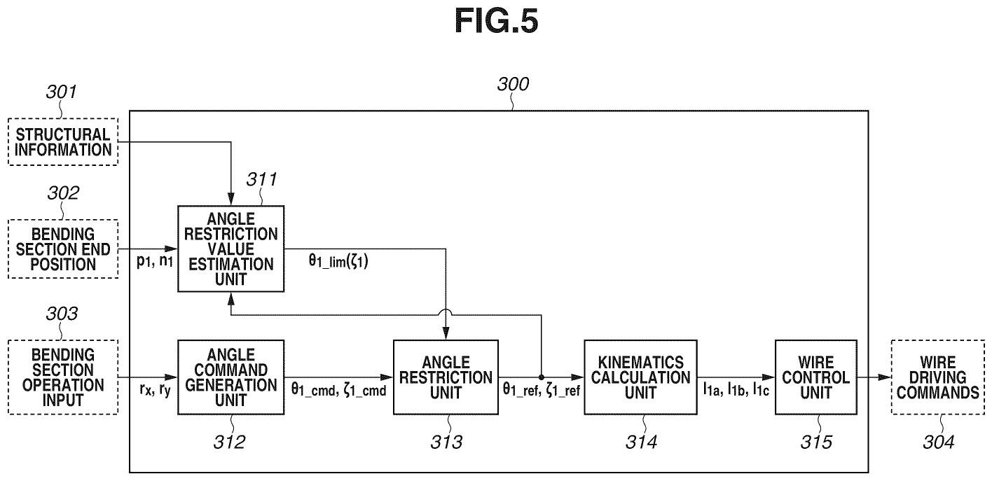

FIG. 5 is a schematic diagram illustrating an example of a general configuration of the control apparatus 300 according to the first exemplary embodiment of the present invention.

The control apparatus 300 illustrated in FIG. 5 includes an angle restriction value estimation unit 311, an angle command generation unit 312, an angle restriction unit 313, a kinematics calculation unit 314, and a wire control unit 315.

In FIG. 5, structural information 301 is structural information about the subject (for example, structural information about the lung's lumen) of the examinee, such as a patient, input from the input device 400 by the user, such as a doctor, before operation. A bending section end position 302 is positional information about the end 120a of the bending section 120, measured using the coil 130. A bending section operation input 303 is input information about the amount of operation when the user, such as a doctor, operates the lever 510 of the operation device 500, for example.

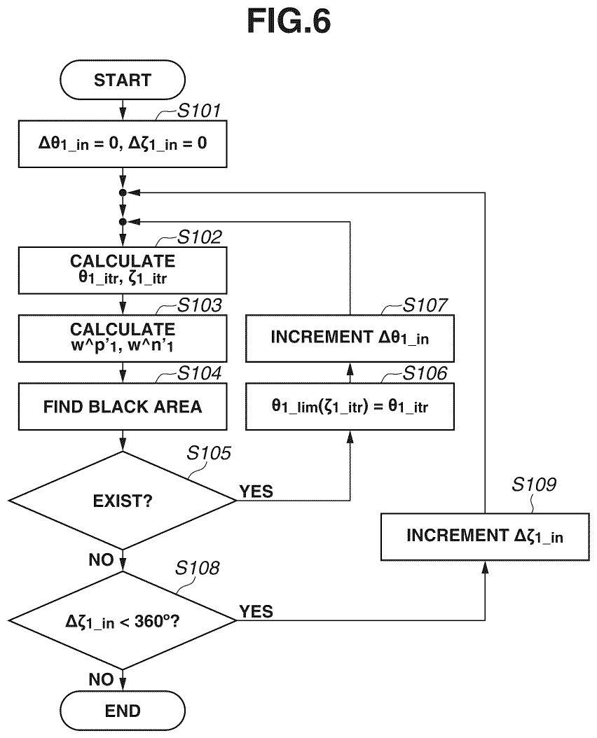

The angle restriction value estimation unit 311 calculates and estimates a bending angle restriction value θ_{1_lim}(ζ₁) for the bending section 120 at a given turning angle (given bending direction) ζ₁ by iterative calculation of FIG. 6 to be described below. Here, the angle restriction value estimation unit 311 calculates and estimates the bending angle restriction value θ_{1_lim}(ζ₁) based on the 3D model including the input structural information 301 about the subject of the examinee, the position p₁ and direction n₁ of the distal end 120a of the bending section 120 included in the input bending section end position 302, and a current target bending angle θ_{1_ref} and target turning angle ζ_{1_ref} output from the angle restriction unit 313.

The angle command generation unit 312 generates by calculation a bending angle command value θ_{1_cmd} and a turning angle command value ζ_{1_cmd} for the bending section 120 based on a lateral tilt amount rₓ and a vertical tilt amount r_{y} of the lever 510, included in the input bending section operation input 303.

The angle restriction unit 313 sets the target bending angle θ_{1_ref} to restrict the driving of the actuators 151a to 153a that are driving units within the range of the bending angle restriction value θ_{1_lim}(ζ₁) for the bending section 120 output from the angle restriction value estimation value 311 (for example, less than or equal to the bending angle restriction value θ_{1_lim}(ζ₁). Specifically, the angle restriction unit 313 initially determines a bending angle restriction value θ_{1_lim}(ζ_{1_cmd}) corresponding to the bending angle command value θ_{1_cmd} output from the angle command generation unit 312. If the bending angle command value θ_{1_cmd} is less than or equal to the bending angle restriction value θ_{1_lim}(ζ_{1_cmd}), the angle restriction unit 313 outputs the bending angle command value θ_{1_cmd} and the turning angle command value ζ_{1_cmd} output from the angle command generation unit 312 as the target bending angle θ_{1_ref} and the target turning angle ζ_{1_ref}, respectively. On the other hand, if the bending angle command value θ_{1_cmd} is greater than the bending angle restriction value θ_{1_lim}(ζ_{1_cmd}), the angle restriction unit 313 simply outputs the current target bending angle θ_{1_ref} without update.

The kinematics calculation unit 314 calculates the driving amounts 1₁ₐ, 1_{1b}, and 1_{1c} of the 1a-, 1b-, and 1c-wires from the target bending angle θ_{1_ref} and the target turning angle ζ_{1_ref} output from the angle restriction unit 313, using the actuator kinematics expressed by Eqs. (1) to (3).

The wire control unit 315 outputs wire driving commands 304 to the actuators 151a to 153a so that the actual driving amounts of the 1a-, 1b-, and 1c-wires match the driving amounts 1₁ₐ, 1_{1b}, and 1_{1c} calculated by the kinematics calculation unit 314.

FIG. 6 is a flowchart illustrating an example of a processing procedure for the iterative calculation by which the angle restriction value estimation unit 311 of FIG. 5 determines the bending angle restriction value θ_{1_lim}(§ 1).

In step S101 of FIG. 6, the angle restriction value estimation unit 311 initializes (sets to zero) a predetermined direction Δζ_{1_in} and a predetermined magnitude Δθ_{1_in} on the assumption that the lever 510 is operated in the predetermined direction Δζ_{1_in} by the predetermined magnitude Δθ_{1_in}.

In step S102, the angle restriction value estimation unit 311 calculates a bending angle θ_{1_itr} and a turning angle ζ_{1_itr} on the assumption that the lever 510 is operated in the predetermined direction Δζ_{1_in} by the predetermined direction Δθ_{1_in}, using a method similar to that of the angle command generation unit 312.

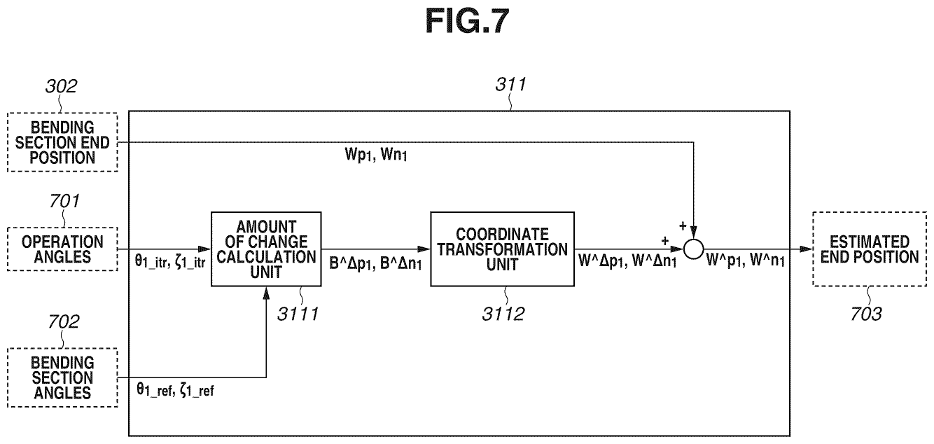

In step S103, the angle restriction value estimation unit 311 calculate a position ^{W}p\hat{ }₁′ and direction ^{W}n\hat{ }₁′ of the end (distal end) 120a of the bending section 120 after the lever 510 is operated, using a calculation method based on the configuration of the block diagram of FIG. 7 to be described below.

In step S104, the angle restriction value estimation unit 311 estimates an image inside the subject (for example, lung) to be output by the imaging unit 140 when the direction indicated by ^{W}n\hat{ }₁′ is observed from the position ^{W}p\hat{ }₁′, using the structural information 301. The angle restriction value estimation unit 311 then determines whether the estimated image includes a part of a path leading to a deep part of the lumen of the subject (for example, lung). Specifically, the angle restriction value estimation unit 311 initially binarizes the estimated image based on brightness so that paths leading to deep parts of the lumen of the subject (for example, lung) are in black and other regions are in white. In step S104, the angle restriction value estimation unit 311 then detects a black area (hereinafter, referred to as “characteristic region”) in the binarized image.

In step S105, the angle restriction value estimation unit 311 determines whether a characteristic region having an area greater than or equal to a predetermined area is detected in step S104. In other words, in step S105, the angle restriction value estimation unit 311 determines whether a characteristic region related to a path of the lumen of the subject (for example, lung) greater than or equal to the predetermined area is included in the field of view of the imaging unit 140 (image obtained by the imaging unit 140).

If, as a result of the determination of step S105, a characteristic region greater than or equal to the predetermined area is detected in step S104 (YES in step S105), the angle restriction value estimation unit 311 determines that a path leading to a deep part of the lumen of the subject (for example, lung) is in the field of view of the imaging unit 140, and the processing proceeds to step S106.

In step S106, the angle restriction value estimation unit 311 updates the bending angle restriction value θ_{1_lim}(ζ_{1_itr}) for the bending section 120 with the bending angle θ₁_itr.

In step S107, the angle restriction value estimation unit 311 increments the predetermined magnitude Δθ_{1_in}. The processing returns to step S102. The processing of steps S102 to S107 is repeated until the binarized image includes no characteristic region greater than or equal to the predetermined area. This can determine θ_{1_lim}(ζ_{1_itr}) that is the limit (for example, upper limit) of the bending angle where a characteristic region greater than or equal to the predetermined area is included in the image at a given operation direction Δζ_{1_in} of the lever 510.

If, as a result of the determination of step S105, no characteristic region greater than or equal to the predetermined area is detected in step S104 (NO in step S105), the processing proceeds to step S108.

In step S108, the angle restriction value estimation unit 311 determines whether the predetermined direction Δζ_{1_in} is less than 360°.

If, as a result of the determination of step S108, the predetermined direction Δζ_{1_in} is less than 360° (YES in step S108), the processing proceeds to step S109.

In step S109, the angle restriction value estimation unit 311 increments the predetermined direction Δζ_{1_in}. The processing returns to step S102.

If, as a result of the determination of step S108, the predetermined direction Δζ_{1_in} is not less than 360° (NO in step S108), the processing of the flowchart of FIG. 6 ends.

By repeating the processing of steps S102 to S109 of FIG. 6 to gradually increase the operation direction (predetermined direction) Δζ_{1_in} of the lever 510 from 0° to 360° while calculating the limit (for example, upper limit) of the bending angle θ_{1_lim}(ζ_{1_itr}) each time, the bending angle restriction value θ_{1_lim}(ζ₁) for the bending section 120 corresponding to all the operation directions can be determined.

FIG. 7 is a schematic diagram illustrating an example of a functional configuration of the angle restriction value estimation unit 311 that performs the processing of step S103 of FIG. 6. Referring to FIG. 7, a method by which the angle restriction value estimation unit 311 calculates estimated values for the position ^{W}p\hat{ }₁′ and direction ^{W}n\hat{ }₁′ of the end (distal end) 120a of the bending section 120 when a predetermined lever operation is assumed in step S103 of FIG. 6 will be described. In FIG. 7, components similar to those illustrated in FIG. 5 are denoted by the same reference numerals. A detailed description thereof will be omitted.

As illustrated in FIG. 7, the angle restriction value estimation unit 311 that performs the processing of step S103 in FIG. 6 includes an amount of change calculation unit 3111 and a coordinate transformation unit 3112 as its functional components.

In FIG. 7, operation angles 701 refer to the bending angle θ_{1_itr} and the turning angle ζ_{1_itr} calculated in step S102 of FIG. 6. The bending section angles 702 refer to the target bending angle θ_{1_ref} and the target turning angle ζ_{1_ref} output from the angle restriction unit 313 of FIG. 5.

The amount of change calculation unit 3111 of FIG. 7 initially calculates a position ^{B}p_{1_ref} and a direction ^{B}n_{1_ref} from the current target bending angle θ_{1_ref} and target turning angle ζ_{1_ref} that are the bending section angles 702, using the robot kinematics expressed by Eqs. (4) and (5). Similarly, the amount of change calculation unit 3111 calculates a position ^{B}p_{1_itr} and a direction ^{B}n_{1_itr} of the distal end 120a after movement from the bending angle θ_{1_itr} and the turning angle ζ_{1_itr} that are the operation angles 701. The amount of change calculation unit 3111 then calculates the amounts of change ^{B}Δ\hat{ }p₁ and βΔ\hat{ }n₁ by subtracting the position ^{B}p_{1_ref} and the direction ^{B}n_{1_ref} before movement from the calculated position ^{B}p_{1_itr} and direction ^{B}n_{1_itr} after movement. The coordinate transformation unit 3112 of FIG. 7 calculates the amounts of change ^{W}Δ\hat{ }p₁ and ^{W}Δ\hat{ }n₁ by transforming the amounts of change ^{B}Δ\hat{ } p₁ and ^{B}Δ\hat{ }n₁ calculated by the amount of change calculation unit 3111 into those of the working coordinate system.

The angle restriction value estimation unit 311 then calculates the position ^{W}p\hat{ }₁′ and the direction ^{W}n\hat{ }₁′ by respectively adding the amounts of change ^{W}Δ\hat{ }p₁ and ^{W}Δ\hat{ }n₁ calculated by the coordinate transformation unit 3112 to the position ^{W}p₁ and direction ^{W}n₁ of the end (distal end) 120a of the bending section 120 measured using the coil 130, i.e., the bending section end position 302. In FIG. 7, the calculated position ^{W}p\hat{ }₁′ and direction ^{W}n\hat{ }₁′ are output as an estimated end position 703.

### [1-4: Processing Procedure for Lung Biopsy]

A processing procedure of a method for restricting the bending angle of the bending section 102 in performing a lung biopsy on an examinee using the foregoing continuum robot control system 10-1 will be described. Before operation, the user generates a 3D model of the lung from medical images of the examinee's lung (subject), such as magnetic resonance imaging (MRI) images and computed tomography (CT) images. The user then determines the target position to sample tissue and the target route for the end 120a of the bending section 120 of the continuum robot 100 to pass through to reach the target position by referring to the generated 3D model. The user then stores the information about the determined target position and target route into a storage unit (not illustrated) of the control apparatus 300 along with the 3D model.

As the operation begins, the user, such as a doctor, initially inserts the imaging tool with the imaging unit 140 at the end into the tool channel 101 of the continuum robot 100, and inserts the imaging unit 140 of the imaging tool up to the end 120a of the bending section 120. The user then inserts the continuum robot 100 with the inserted imaging tool from the mouth or nose of the examinee. The user then moves forward the linear stage 200 on which the driving unit 150 of the continuum robot 100 is mounted while referring to the camera image 610 and the navigation image 620 displayed on the image display device 600 and operating the operation device 500 (such as the lever 510) to control the orientation of the end 120a of the bending section 120.

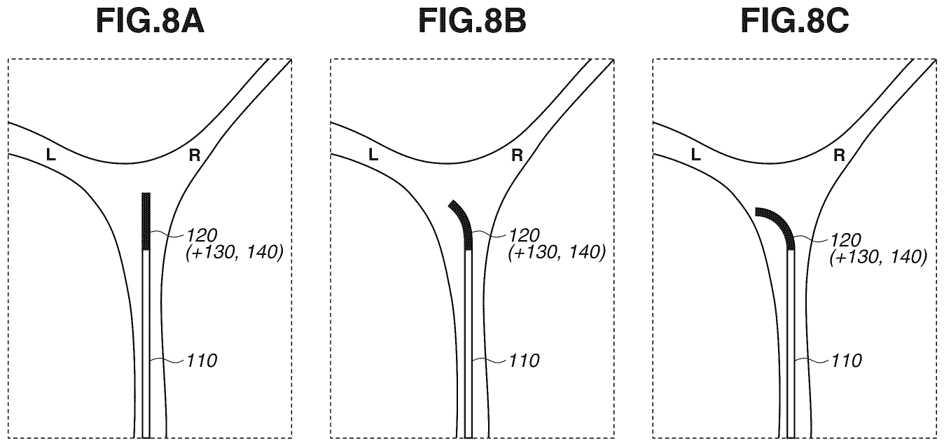

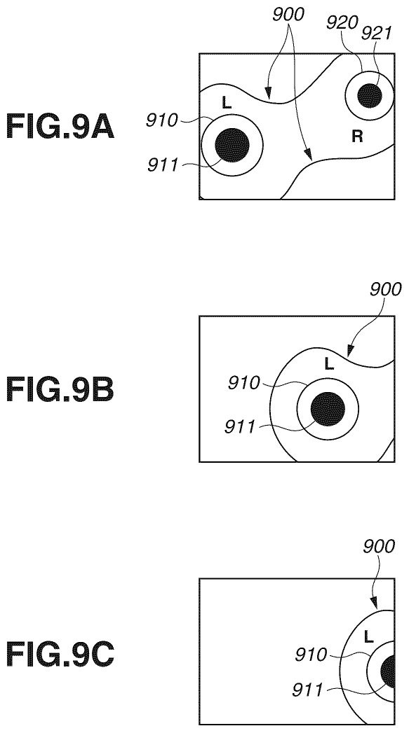

FIGS. 8A, 8B, and 8C are diagrams illustrating the first exemplary embodiment of the present invention, illustrating examples of the orientation of the continuum robot 100 inside the subject. In FIGS. 8A, 8B, and 8C, components similar to those illustrated in FIGS. 1 to 4 are denoted by the same reference numerals. A detailed description thereof will be omitted. FIGS. 9A, 9B, and 9C are diagrams illustrating the first exemplary embodiment of the present invention, illustrating examples of the camera image output by the imaging unit 140 when the continuum robot 100 assumes the orientations of FIGS. 8A, 8B, and 8C. The following description will be given with reference to FIGS. 8A, 8B, 8C, 9A, 9B, and 9C.

Initially, as illustrated in FIG. 8A, the end 120a of the bending section 120 where the coil 130 and the imaging unit 140 are located reaches the vicinity of a branch in the lumen of the lung that is the subject. Here, as illustrated in FIG. 9A, the field of view (camera image) of the imaging unit 140 includes both a path L (910) of FIG. 8A on the left part of the screen and a path R (920) of FIG. 8A on the right part of the screen. FIG. 9A also illustrates a Olumen wall (intraluminal wall) 900, a path 911 of the lumen leading to a deep part of the path L 910, and a path 921 of the lumen leading to a deep part of the path R 920, for example. The paths 911 and 912 correspond to the foregoing characteristic region.

Next, as illustrated in FIG. 8B, the bending section 120 with the coil 130 and the imaging unit 140 at the end 120a is bent to the left (predetermined direction). This moves the path L 910 and the path R 920 to the right onscreen. FIG. 9B illustrates the field of view (camera image) of the imaging unit 140 here. The path R 920 goes off the screen, but the path L 910 including the lumen path 911 corresponding to the characteristic region remains within the screen. In the present exemplary embodiment, the angle restriction unit 313 therefore does not restrict the bending angle of the bending section 120.

Next, as illustrated in FIG. 8C, the bending section 120 with the coil 130 and the imaging unit 140 at the end 120a is further bent to the left to increase the bending angle. FIG. 9C illustrates the field of view (camera image) of the imaging unit 140 here. A part of the path L 910 falls outside the screen, and the area of the lumen path 911 corresponding to the characteristic region decreases. In the present exemplary embodiment, if the area of the lumen path 911 corresponding to the characteristic region falls below a predetermined area that is a constant threshold, the control apparatus 300 (angle restriction unit 313) controls the bending section 120 to not bend to the left (predetermined direction) any further despite the user's bending section operation input 303 to further bend the bending section 120 to the left. Note that the angle restriction unit 313 stores different bending angle restriction values θ_{1_lim}(ζ) for respective turning angles ζ. The angle restriction unit 313 does not restrict the bending angle of the bending section 120 if a bending section operation input 303 upward, downward, or to the right (direction other than the predetermined direction) onscreen is given to increase the area of the foregoing characteristic region.

As described above, the control by the control apparatus 300 according to the present exemplary embodiment keeps the characteristic region related to the lumen path of the subject displayed on the camera image 610. The user can thus easily find out the operation direction of the bending section 120. This can reduce the risk of accidentally operating the bending section 120 in a direction of causing strong contact with the lumen of the subject and damaging the continuum robot 100.

In the present exemplary embodiment, the area of the characteristic region is calculated based on a difference in brightness between the lumen wall of the lung that is the subject and the path leading to the deep part of the lumen wall of the lung. However, the method by which the control apparatus 300 according to the present exemplary embodiment calculates the characteristic region is not limited thereto. In the present exemplary embodiment, information about other than the lumen may be used as long as the characteristic region can be identified from the background. For example, the characteristic region may be defined using a difference in brightness due to irregularities of the lumen wall of the subject, such as tumors and folds, or information about edges occurring from branching or bending of the lumen of the subject.

In the present exemplary embodiment, the area of the characteristic region is calculated using the camera image estimated by the angle restriction value estimation unit 311. However, an image obtained by applying image processing such as transparency processing to the structural information about the tissue of the subject may be used. This enables use of lumens and irregularities behind the lumen wall on the foreground as the characteristic region, for example. As compared to the case of estimating the camera image, the user's operability of the bending section 120 can thus be improved by increasing the bending angle restriction value.

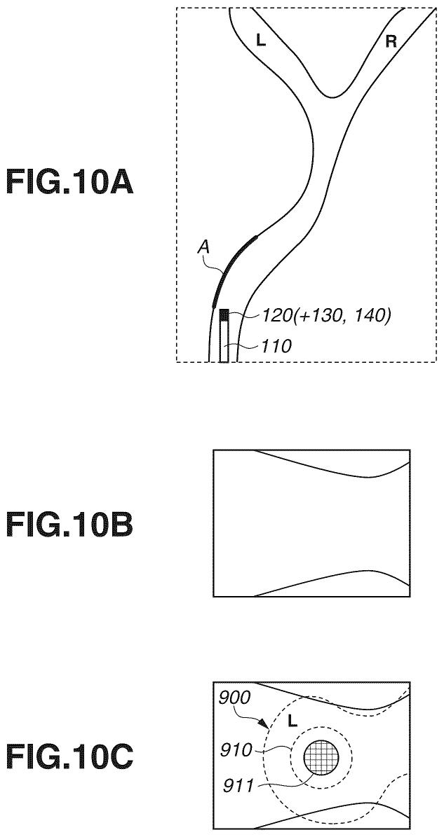

FIGS. 10A, 10B, and 10C are diagrams illustrating an example of a mode where the bending angle restriction value for the bending section 120 of the continuum robot 100 according to the first exemplary embodiment of the present invention can be increased. In FIGS. 10A, 10B, and 10C, components similar to those illustrated in FIGS. 8A, 8B, 8C, 9A, 9B, and 9C are denoted by the same reference numerals. A detailed description thereof will be omitted.

FIG. 10A is a diagram illustrating an example of the orientation of the continuum robot 100 inside the subject. In the interior of the subject illustrated in FIG. 10A, the path L continues behind a lumen wall A when seen from the continuum robot 100. As illustrated in FIG. 10B, the field of view (camera image) of the imaging unit 140 here includes only the lumen wall A without the foregoing characteristic region. By using only the camera image, the bending section 120 is therefore difficult to operate into the orientation illustrated in FIG. 10A. As illustrated in FIG. 10C, an image including the path L can be generated, however, by making the lumen wall A transparent. If such a transparent image is used as the navigation image 620, or the path L 910 extracted from the transparent image by image processing is superimposed on the camera image 610, the user can determine the operation direction of the bending section 120 by referring to the information about the path L 910, and identify the path L 910 as a characteristic region 911. In such a manner, the bending angle restriction value for the bending section 120 can be increased so that the bending section 120 assumes the orientation illustrated in FIG. 10A.

Moreover, the foregoing characteristic region may be set based on the target route up to the affected area (region of interest) inside the subject, planned before operation. For example, the information about the target route is superposed on the 3D model of the tissue, and the camera image 610 is estimated to include the target route. The bending angle restriction value for the bending section 120 may then be calculated so that a part of the target route is always included in the estimated camera image 610.

The control apparatus 300 of the continuum robot control system 10-1 according to the first exemplary embodiment performs the following processing.

Using the angle restriction value estimation unit 311 (angle estimation unit), the control apparatus 300 initially estimates the bending angle restriction value θ_{1_lim}(ζ₁) for the bending section 120 for a case where the bending section 120 is bent in a predetermined direction and a characteristic region related to the path of the lumen greater than or equal to a predetermined area is included in the field of view of the imaging unit 140 based on the bending section end position 302 detected after the insertion of the bending section 120 into the lumen of the subject (for example, the examinee's lung) and the structural information 301 about the lumen. Using the angle restriction unit 313 (angle restriction unit), the control apparatus 300, in bending the bending section 120 in the predetermined direction, restricts the driving of the actuators 151a to 153a that are the driving sections so that the bending section 120 bends within the range of the bending angle restriction value θ_{1_lim}(ζ₁).

With such a configuration, the bending angle of the bending section 120 can be controlled so that the characteristic region related to the lumen path of the subject is always included in the field of view of the imaging unit 140. This can reduce the risk of operating the continuum robot 100 in a direction of causing strong contact with the lumen of the subject, even if the subject has a small diameter and changes in shape during operation, such as bronchi.

The first exemplary embodiment also includes the processing method (continuum robot control method) performed by the continuum robot control system 10-1.

Next, a second exemplary embodiment of the present invention will be described. In the following description of the second exemplary embodiment, a description of matters common with the foregoing first exemplary embodiment will be omitted, and differences from the foregoing first exemplary embodiment will be described.

As described in the first exemplary embodiment, the continuum robot 100 is fixed to the linear stage 200. The area of the characteristic region on the camera image 610 therefore changes not only with the bending operation of the bending section 120 but also with the forward and backward motion of the linear stage 200. A control apparatus 300 according to the present exemplary embodiment therefore also restricts the amount of movement of the linear stage 200 so that the foregoing characteristic region has a predetermined area or more.

### [2-1: Configuration of Continuum Robot Control System]

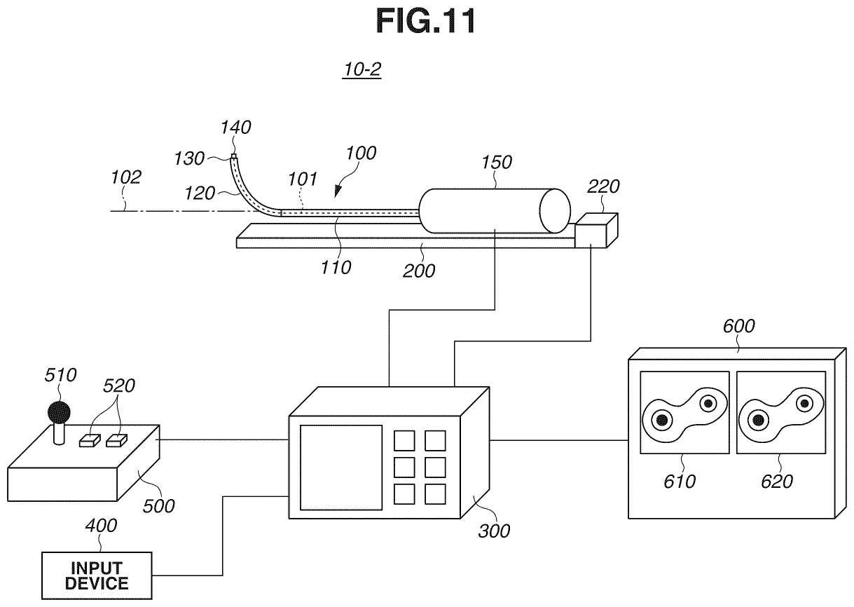

FIG. 11 is a schematic diagram illustrating an example of a general configuration of a continuum robot control system 10-2 according to the second exemplary embodiment of the present invention. In FIG. 11, components similar to those illustrated in FIG. 1 are denoted by the same reference numerals. A detailed description thereof will be omitted.

As illustrated in FIG. 11, the continuum robot control system 10-2 includes the continuum robot 100, the linear stage 200, an electric stage 220, the control apparatus 300, the input device 400, the operation device 500, and the image display device 600.

In the second exemplary embodiment, the linear stage 200 is driven by the electric stage 220 including an electric actuator. The linear stage 200 and the electric stage 220 correspond to a moving device that moves the continuum robot 100 forward and backward with respect to the subject of the examinee. The operation device 500 according to the second exemplary embodiment includes forward/backward buttons 520 (forward button and backward button) for issuing forward and backward movement commands to the electric stage 220. In the present exemplary embodiment, when the user presses the forward/backward buttons 520, the control apparatus 300 outputs a driving command to the electric actuator of the electric stage 200 based on the type of pressed button. The rotational motion of the electric actuator is converted into translational motion by a feed screw, and the driving unit 150 makes a forward or backward motion (forward/backward movement) along with the table of the electric stage 220. A not-illustrated encoder is connected to the electric actuator of the electric stage 220. The control apparatus 300 calculates the amount of movement of the table (stage) based on the output of the encoder.

### [2-2: Configuration of Control Apparatus]

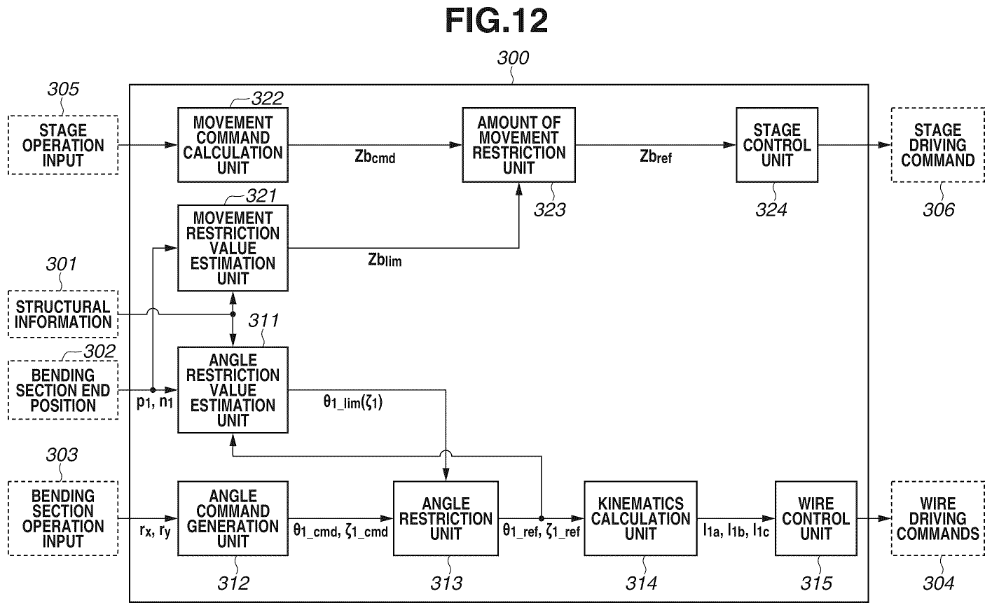

FIG. 12 is a schematic diagram illustrating an example of a general configuration of the control apparatus 300 according to the second exemplary embodiment of the present invention. In FIG. 12, components similar to those illustrated in FIG. 5 are denoted by the same reference numerals. A detailed description thereof will be omitted.

The control apparatus 300 illustrated in FIG. 12 includes a movement restriction value estimation unit 321, a movement command calculation unit 322, an amount of movement restriction unit 323, and a stage control unit 324 in addition to the components 311 to 315 illustrated in FIG. 5. The components 321 to 314 are ones related to the control system of the electric stage 220.

In FIG. 12, the structural information 301, the bending section end position 302, and the bending section operation input 303 are similar to those of FIG. 5. In FIG. 12, a stage operation input 305 is input information about the type of button and the amount of operation when the user, such as a doctor, operates the forward/backward buttons 520 of the operation device 500, for example.

The movement restriction value estimation unit 321 calculates and estimates a movement restriction value zbₗᵢₘ for the amount of movement of the stage based on the input structural information 301 and the position p₁ and direction n₁ of the distal end of the bending section 120 included in the bending section end position 302, using iterative calculation like the angle restriction value estimation unit 311 described in the first exemplary embodiment. Specifically, the movement restriction value estimation unit 321 initially estimates the position and direction of the imaging unit 140 when the stage is moved by a predetermined amount, and estimates an image to be output by the imaging unit 140 based on the estimated position and direction and the structural information 301. The movement restriction value estimation unit 321 then determines whether the characteristic region included in the estimated image is greater than or equal to a predetermined area.

The movement restriction value estimation unit 321 repeats the foregoing processing while increasing the amount of movement of the stage, and outputs the maximum amount of movement of the stage at which the characteristic region is included in the estimated image as the movement restriction value zbₗᵢₘ.

The movement command calculation unit 322 calculates a position command value zb_{cmd} for the stage based on the stage operation input 305.

If the position command value zb_{cmd} calculated by the movement command calculation unit 322 is less than or equal to the movement restriction value zbₗᵢₘ, the amount of movement restriction unit 323 outputs the position command value zb_{cmd} as a target position zb_{ref}. If the position command value zb_{cmd} calculated by the movement command calculation unit 322 is greater than the movement restriction value zbₗᵢₘ, the amount of movement restriction unit 323 outputs the movement restriction value zbₗᵢₘ as the target position zb_{ref}.

The stage control unit 324 outputs a stage driving command 306 so that the position of the stage measured by the encoder connected to the electric actuator of the electric stage 220 matches the target position zb_{ref}.

### [2-3: Processing Procedure for Lung Biopsy]

A processing procedure of a method for controlling the electric stage 220 during a lung biopsy operation on the examinee using the foregoing continuum robot control system 10-2 will be described.

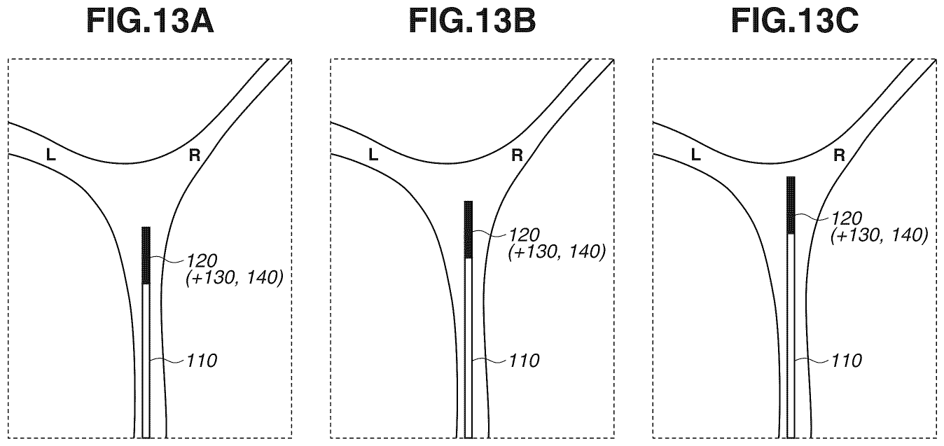

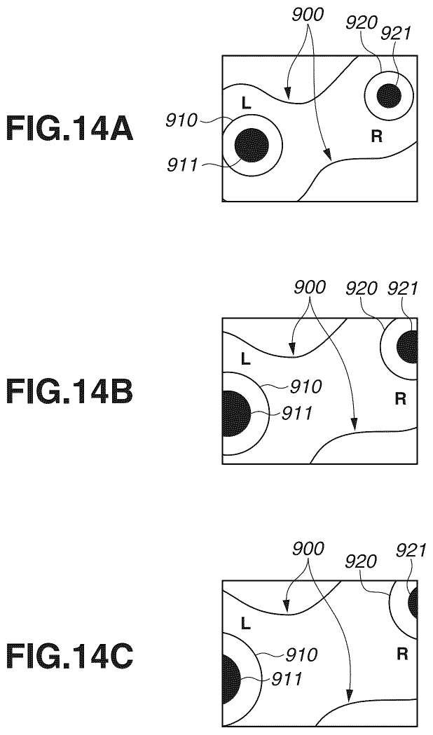

FIGS. 13A, 13B, and 13C are diagrams illustrating the second exemplary embodiment of the present invention, illustrating examples of the orientation of the continuum robot 100 inside the subject. In FIGS. 13A, 13B, and 13C, components similar to those illustrated in FIGS. 1 to 4, 8A, 8B, 8C, and 11 are denoted by the same reference numerals. A detailed description thereof will be omitted. FIGS. 14A, 14B, and 14C are diagrams illustrating the second exemplary embodiment of the present invention, illustrating examples of the camera image output by the imaging unit 140 when the continuum robot 100 assumes the orientation of FIGS. 13A, 13B, and 13C. In FIGS. 14A, 14B, and 14C, components similar to those illustrated in FIGS. 9A, 9B, and 9C are denoted by the same reference numerals. A detailed description thereof will be omitted. The method for controlling the electric stage 220 during the lung biopsy operation on the examinee will now be described with reference to FIGS. 13A, 13B, 13C, 14A, 14B, and 14C.

Initially, as illustrated in FIG. 13A, the end of the bending section 120 where the coil 130 and the imaging unit 140 are located reaches the vicinity of a branch in the lumen of the lung that is the subject. As illustrated in FIG. 14A, the field of view (camera image) of the imaging unit 140 here includes a path L (910) of FIG. 13A on the left part of the screen and a path R (920) of FIG. 13A on the right part of the screen.

When the stage is moved forward, the end of the bending section 120 where the coil 130 and the imaging unit 140 are located approaches the branch as illustrated in FIG. 13B. Here, as illustrated in FIG. 14B, the path L 910 moves to the left and the path R 920 moves to the right in the field of view (camera image) of the imaging unit 140.

When the stage is further moved forward, the end of the bending section 120 where the coil 130 and the imaging unit 140 are located further approaches the branch as illustrated in FIG. 13C. FIG. 14C illustrates the field of view (camera image) of the imaging unit 140 here. Most of the path L 910 and the path R 920 fall outside the camera image 610, and lumen paths 911 and 921 corresponding to characteristic regions become smaller than a predetermined area. The amount of movement restriction unit 323 therefore controls so as to restrict the forward movement of the stage in such a case.

As illustrated in FIG. 12, the amount of movement restriction unit 323 for the stage and the angle restriction unit 313 for the bending angle of the bending section 120 are independent of each other. This enables the bending operation of the bending section 120 even if the movement of the stage is restricted. For example, in the case where the camera image illustrated in FIG. 14C is obtained, bending the bending section 120 to the left moves the path L 910 toward the center of the camera image, and bending the bending section 120 to the right moves the path R 920 toward the center of the camera image. The bending angle of the bending section 120 can thus be left unrestricted. If the area of a characteristic region reaches or exceeds the predetermined area as a result of such a bending operation, the restriction on the amount of movement of the stage is lifted and the stage can be moved forward again.

The control apparatus 300 of the continuum robot control system 10-2 according to the second exemplary embodiment performs the following processing in addition to the processing of the control apparatus 300 according to the first exemplary embodiment.

Using the movement restriction value estimation unit 321 (movement estimation unit), the control apparatus 300 initially estimates the movement restriction value zbₗᵢₘ for the amount of movement of the linear stage 200 and the electric stage 220 that are moving devices based on the bending section end position 302 detected after the insertion of the bending section 102 into the lumen of the subject (for example, the examinee's lung) and the structural information 301 about the lumen. Using the amount of movement restriction unit 323 (amount of movement restriction unit), the control apparatus 300 then restricts the amount of movement of the linear stage 200 and the electric stage 220 that are the moving devices within the range of the movement restriction value zbₗᵢₘ.

With such a configuration, the stage position can be controlled to prevent the continuum robot 100 from moving so much with respect to the subject so that the characteristic region falls outside the field of view (camera image 610) of the imaging unit 140.

In the present exemplary embodiment, the amount of movement of the stage is restricted by using the electric actuator of the electric stage 220. However, the present exemplary embodiment can be applied to other exemplary embodiments as well. For example, a continuum robot control system may include a stage that the user can manually operate to move forward like the first exemplary embodiment, an encoder that measures the amount of movement of the stage, and an electromagnetic brake that restricts the forward and backward movement of the stage. In such a configuration, when the stage is moved by the user operation and the amount of movement zb measured by the encoder reaches or exceeds the target position zbₗᵢₘ, the electromagnetic brake can be applied to keep the amount of movement within the target value zbₗᵢₘ.

Next, a third exemplary embodiment of the present invention will be described. In the following description of the third exemplary embodiment, a description of matters common with the foregoing first and second exemplary embodiments will be omitted, and differences from the foregoing first and second exemplary embodiments will be described.

As described in the first exemplary embodiment, the elongated section 110 of the continuum robot 100 can be passively bent when the elongated section 110 contacts the lumen of the subject. However, when passing through a path with a large curvature, the elongated section 110 may strongly contact the lumen of the subject. In view of this, a continuum robot with a plurality of bending sections 120 may be used to actively control the orientation of each bending section 120 along the shape of the lumen of the subject. This can reduce the risk of deterioration in operability or damage to the continuum robot 100 due to contact between the subject's lumen and the continuum robot 100. The third exemplary embodiment thus applies a continuum robot 100 including a plurality of bending sections 120, and the bending angles of the bending sections 120 are restricted to keep the foregoing characteristic region within the camera image 610 even when the bending sections 120 other than the one at the end (distal end) are operated.

### [3-1: Configuration of Continuum Robot Control System]

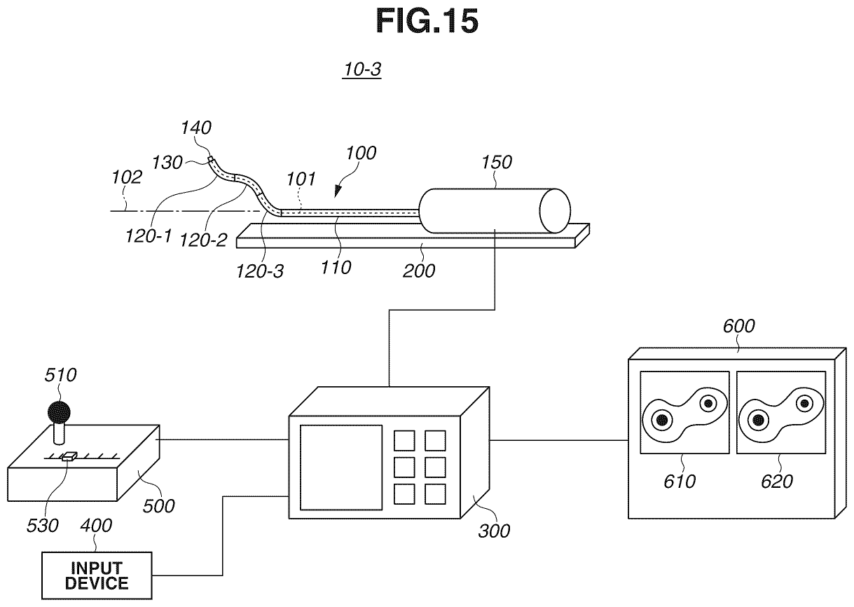

FIG. 15 is a schematic diagram illustrating an example of a general configuration of a continuum robot control system 10-3 according to the third exemplary embodiment of the present invention. In FIG. 15, components similar to those illustrated in FIGS. 1 and 11 are denoted by the same reference numerals. A detailed description thereof will be omitted.

The continuum robot control system 10-3 according to the third exemplary embodiment differs from the foregoing first and second exemplary embodiments in that the continuum robot 100 includes a plurality of bending sections 120-1 to 120-3. The user, such as a doctor, can change the bending angle and orientation of one of the plurality of bending sections 120-1 to 120-3 by using the lever 510 on the operation device 500 as with the continuum robot control system 10-1 according to the first exemplary embodiment. In the continuum robot control system 10-3 according to the third exemplary embodiment, the operation device 500 include a slide switch 530. The user can select the bending section 120 to operate by changing the position of the slider on the slide switch 530.

### [3-2: Configuration of Continuum Robot]

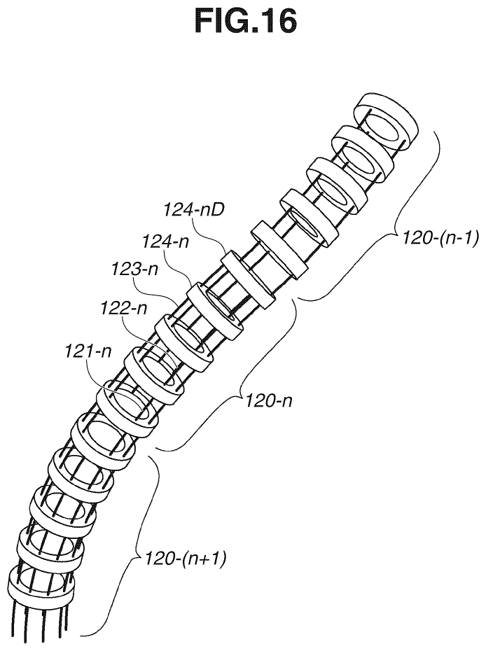

FIG. 16 is a schematic diagram illustrating an example of the plurality of bending sections 120 included in the continuum robot 100 according to the third exemplary embodiment of the present invention. In FIG. 16, components similar to those illustrated in FIGS. 2 to 4 and 15 are denoted by the same reference numerals. A detailed description thereof will be omitted.

In the present exemplary embodiment, the number of bending sections 120 is N. In FIG. 16, one of the N bending sections 120 is illustrated as an nth (n=1, 2, . . . , N) bending section 120-n. The nth bending section 120-n includes a plurality of wire guides 124-n. Drive wires 121-n, 122-n, and 123-n are fixedly connected at one end to a wire guide 124nD located at the distal end of the plurality of wire guides 124-n. In the following description, the drive wire 121-n illustrated in FIG. 16 will be referred to as an “na-wire”, the drive wire 122-n illustrated in FIG. 16 as an “nb-wire”, and the drive wire 123-n illustrated in FIG. 16 as an “nc-wire”. Only the na-wire is fixed to the wire guides 124-n other than the wire guide 124-nD at the distal end of the nth bending section 120-n. The nb- and nc-wires can slide longitudinally through not-illustrated guide holes in the wire guides 124-n.

FIG. 16 illustrates three bending sections 120, namely, an (n−1)th bending section 120-(n−1), the nth bending section 120-n, and an (n+1)th bending section 120-(n+1) in order from the end of the continuum robot 100. In such a case, the (n−1)th bending section 120-(n−1) illustrated in FIG. 16 corresponds to the first bending section 120-1 illustrated in FIG. 15. Similarly, the nth bending section 120-n illustrated in FIG. 16 corresponds to the second bending section 120-2 illustrated in FIG. 15. The (n+1)th bending section 120-(n+1) illustrated in FIG. 16 corresponds to the third bending section 120-3 illustrated in FIG. 15.

In FIG. 16, none of the na-, nb-, and nc-wires is fixed to the wire guides of the (n+1)th bending section 120-(n+1) located on the bottom side of the nth bending section 120-n. The na-, nb-, and nc-wires slide through guide holes in those wire guides. The wires having passed through the (n+1)th bending section 120-(n+1) corresponding to the Nth bending section located at the bottommost side are guided through the elongated section 110 and connected to respective actuators that are not-illustrated driving sections. Driving the respective actuators push or pull the na-, nb-, and nc-wires, whereby a bending angle θₙ and a turning angle ζₙ of the nth bending section 120-n are changed. The actuator kinematics representing the relationship between the bending angle θₙ and turning angle ζₙ of the nth bending section 120-n and the driving amounts Iₙₐ, I_{nb}, and I_{nc} of the na-, nb-, and nc-wires is expressed by the following Eqs. (6) to (8):

$${l}_{na} = \frac{{R}_{g} {θ}_{n}}{2} cos ( {ξ}_{n} - {ζ}_{n} ) \qquad ( 6 )$$

$${l}_{nb} = \frac{{R}_{g} {θ}_{n}}{2} cos ( {ξ}_{n} - {ζ}_{n} + \frac{2 π}{3} ) \qquad ( 7 )$$

$${l}_{nc} = \frac{{R}_{g} {θ}_{n}}{2} cos ( {ξ}_{n} - {ζ}_{n} + \frac{4 π}{3} ) \qquad ( 8 )$$

As described above, the na-, nb-, and nc-wires are not connected to any wire guide other than those of the nth bending section 120-n. The bending angles and turning angles of the bending sections 120 other than the nth bending section 120-n therefore remain unchanged if the driving amounts Iₙₐ, I_{nb}, and I_{nc} are changed. In such a manner, the continuum robot 100 according to the present exemplary embodiment can independently control the orientation of each bending section 120.

The robot kinematics representing the angle and the position ^{B}p₁ of the end of the first bending section 120-1 can be determined using the kinematics of each bending section 120. The control apparatus 300 initially determines a vector from the bottommost wire guide to the distal end of each bending section 120 using an equation similar to Eq. (4). The control apparatus 300 then transforms the respective vectors into coordinate systems with the bases of the respective bending sections 120 as origins and the coordinate axes X_{B}, Y_{B}, and Z_{B} of the robot coordinate system as coordinate axes. The control apparatus 300 then adds the resulting vectors to determine the position ^{B}p₁. Since the direction n₁ of the end of the first bending section 120-1 depends only on the bending angle θ₁ and the turning angle ζ₁ of the first bending section 120-1, the direction n₁ of the continuum robot 100 according to the present exemplary embodiment can also be determined using Eq. (5).

### [3-3: Control of Control Apparatus]

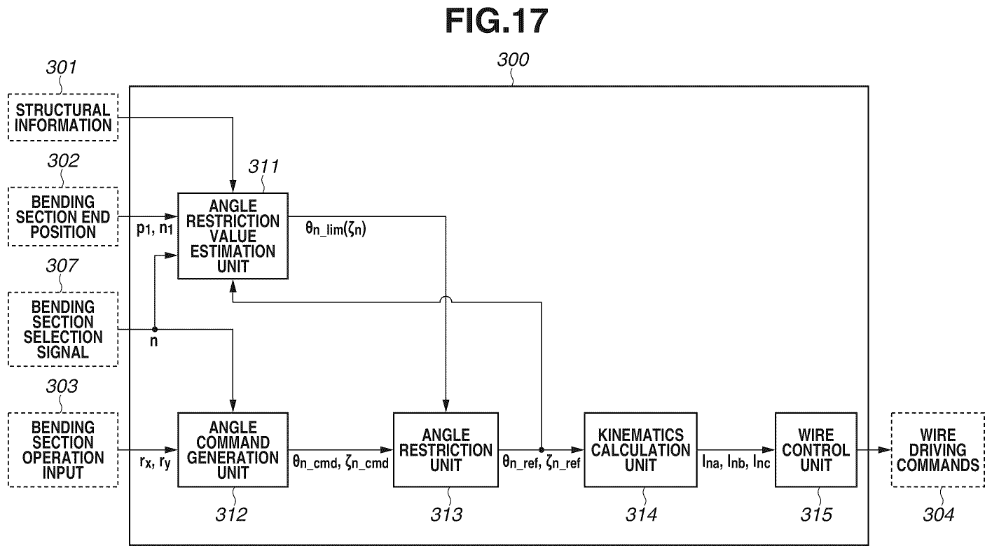

FIG. 17 is a schematic diagram illustrating an example of a general configuration of the control apparatus 300 according to the third exemplary embodiment of the present invention. In FIG. 17, components similar to those illustrated in FIGS. 5 and 12 are denoted by the same reference numerals. A detailed description thereof will be omitted.

Like the control apparatus 300 illustrated in FIG. 5, the control apparatus 300 illustrated in FIG. 17 includes the angle restriction value estimation unit 311, the angle command generation unit 312, the angle restriction unit 313, the kinematics calculation unit 314, and the wire control unit 315.

In FIG. 17, the structural information 301, the bending section end position 302, and the bending section operation input 303 are similar to those of FIG. 5. In FIG. 17, a bending section selection signal 307 is a selection signal indicating a bending section 120 that is selected by the user, such as a doctor, operating the slide switch 530 of the operation device 500, for example.

In the third exemplary embodiment, the angle restriction value estimation unit 311 estimates the image to be output by the imaging unit 140 when the nth bending section 120-n is driven, based on the input structural information 301, the position p₁ and direction n₁ of the distal end of the first bending section 120-1 included in the input bending section end position 302, the number n of the bending section 120-n to be operated indicated by the bending section selection signal 307, and a target bending angle θ_{n_ref} and a target turning angle ζ_{n_ref} of the nth bending section 120-n output from the angle restriction unit 313. The angle restriction value estimation unit 311 then outputs the maximum bending angle where a characteristic region greater than or equal to a predetermined area is included in the estimated image as a bending angle restriction value θ_{n_lim}(ζₙ) for the nth bending section 120-n through the iterative calculation described in the first exemplary embodiment.

In the third exemplary embodiment, the angle command generation unit 312 generates a bending angle command value θ_{n_cmd} and a turning angle command value ζ_{n_cmd} for the nth bending section 120-n by calculation based on the lateral tilt amount rₓ and the vertical tilt amount r_{y} of the lever 510 included in the input bending section operation input 303, and the number n of the bending section 120 to be operated indicated by the bending section selection signal 307.

In the third exemplary embodiment, the angle restriction unit 313 sets a target bending angle θ_{n_ref} to restrict the driving of the actuators that are the driving sections within the range of the bending angle restriction value θ_{n_lim}(ζₙ) for the nth bending section 120-n output from the angle restriction value estimation unit 311 (for example, less than or equal to the bending angle restriction value θ_{1_lim}(ζ₁).

In the third exemplary embodiment, the kinematics calculation unit 314 calculates the driving amounts 1ₙₐ, 1_{nb}, and 1_{nc} of the na-, nb-, and nc-wires of the nth bending section 120-n from the target bending angle θ_{n_ref} and the target turning angle ζ_{n_ref} output from the angle restriction value 313, using the kinematics expressed by Eqs. (6) to (8).

In the third exemplary embodiment, the wire control unit 315 outputs wire driving commands 304 to the respective actuators so that the actual driving amounts of the na-, nb-, and nc-wires match the driving amounts Iₙₐ, I_{nb}, and I_{nc} calculated by the kinematics calculation unit 314, respectively.

The control apparatus 300 of the continuum robot control system 10-3 according to the third exemplary embodiment performs the following processing on the continuum robot 100 including the plurality of bending sections 120.

Using the angle restriction value estimation unit 311 (angle estimation unit), the control apparatus 300 initially estimates the bending angle restriction value θ_{n_lim}(ζₙ) for the nth bending section 120-n for a case where a characteristic region related to the lumen path and greater than or equal to a predetermined area is included in the field of view of the imaging unit 140 in bending the nth bending section 120-n in a predetermined direction. Here, the angle restriction value estimation unit 311 calculates the bending angle restriction value θ_{n_lim}(ζₙ) based on the end position of the distal bending section 120-1 detected after the insertion of the plurality of bending sections 120 into the lumen of the subject (for example, the examinee's lung), the structural information 301 about the lumen, and the bending section selection signal 307 (selection information) indicating the nth bending section 120-n selected as the bending section 120 to be operated. Using the angle restriction unit 313 (angle restriction unit), the control apparatus 300, in bending the nth bending section 120-n in the predetermined direction, restricts the driving of the actuators that are the driving sections so that the nth bending section 120-n bends within the range of the bending angle restriction value θ_{n_lim}(ζn) for the nth bending section 120-n.

With such a configuration, even if the continuum robot 100 includes the plurality of bending sections 120, the characteristic region can be prevented from falling outside the field of view (camera image 610) of the imaging unit 140. This can reduce the risk of operating the continuum robot 100 in a direction of causing strong contact with the lumen of the subject.

### Other Exemplary Embodiments

In the foregoing first to third exemplary embodiments, the lung of the examinee, such as a patient, is described to be assumed as the subject to insert the bending section(s) 120 of the continuum robot 100 into. However, the present invention is not limited thereto. Organs (viscera) other than the lung that have lumens may be applied as the subject to insert the bending section(s) 120 of the continuum robot 100 into. In the foregoing first to third exemplary embodiments, the examinee, such as a patient, is described to be assumed as the target to insert the bending section(s) 120 of the continuum robot 100 into. However, the present invention is not limited thereto. The target may be animals other than humans. In other words, an exemplary embodiment of the present invention covers the application of organs (viscera) with lumens of animals other than humans as subjects to insert the bending section(s) 120 of the continuum robot 100 into.

An exemplary embodiment of the present invention can also be achieved by processing for supplying a program for implementing one or more of the functions of the foregoing exemplary embodiments to a system or an apparatus via a network or a storage medium, and reading and executing the program by one or more processors in a computer of the system or apparatus. A circuit that implements one or more functions (for example, an application-specific integrated circuit [ASIC]) may be used for implementation.

The program and a computer-readable storage medium storing the program are also included in the present invention.

All the foregoing exemplary embodiments of the present invention are merely examples of embodiment in carrying out the present invention, and should not be interpreted as limiting the technical scope of the present invention. In other words, the present invention can be implemented in various forms without departing from the technical concept or main features thereof.

The present invention is not limited to the foregoing exemplary embodiments, and various changes and modifications can be made without departing from the spirit and scope of the present invention. The following claims are therefore attached to disclose the scope of the present invention.

According to an exemplary embodiment of the present invention, the risk of operating a continuum robot in a direction of causing strong contact with the lumen of a subject can be reduced.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A continuum robot control system comprising:
a continuum robot including a bending section configured to be bent with respect to a reference axis by a linear member being driven, a driving unit configured to drive the linear member, and an imaging unit located near an end of the bending section; and
a control apparatus configured to control operation of the continuum robot,
the control apparatus including
an angle estimation unit configured to, in a case where a characteristic region related to a path of a lumen of a subject greater than or equal to a predetermined area is included in a field of view of the imaging unit in bending the bending section in a predetermined direction, estimate an angle restriction value for a bending angle of the bending section based on an end position of the bending section detected after insertion of the bending section into the lumen and structural information about the lumen, and
an angle restriction unit configured to, in bending the bending section in the predetermined direction, restrict driving of the driving unit so that the bending section bends within a range of the angle restriction value.

2. The continuum robot control system according to claim 1, wherein the angle estimation unit is configured to estimate the angle restriction value by determining a position and direction of the end of the bending section in a case where the bending section bends in the predetermined direction by a predetermined bending angle, estimating an image to be obtained by the imaging unit based on the determined position and direction of the end of the bending section and the structural information about the lumen, and determining whether the characteristic region is included in the estimated image.

3. The continuum robot control system according to claim 1, further comprising a moving device configured to move the continuum robot forward and backward with respect to the subject,
wherein the control apparatus further includes
a movement estimation unit configured to estimate a movement restriction value for an amount of movement by the moving device based on the end position of the bending section detected after the insertion of the bending section into the lumen and the structural information about the lumen, and
an amount of movement restriction unit configured to restrict the amount of movement by the movement device within a range of the movement restriction value.

4. The continuum robot control system according to claim 1,
wherein the continuum robot includes a plurality of bending sections,
wherein the angle estimation unit is configured to, in a case where the characteristic region greater than or equal to the predetermined area is included in the field of view of the imaging unit in bending one of the bending sections in a predetermined direction, estimate the angle restriction value for the bending angle of the one bending section based on an end position of a distal bending section among the plurality of bending sections, the structural information about the lumen, and selection information about the one bending section among the plurality of bending sections, the end position being detected after insertion of the plurality of bending sections into the lumen, and
wherein the angle restriction unit is configured to, in bending the one bending section in the predetermined direction, restrict the driving of the driving unit so that the one bending section bends within a range of the angle restriction value for the one bending section.

5. The continuum robot control system according to claim 1,
wherein the continuum robot further includes a tool channel configured to insert and remove a tool, the tool channel being a tube-like passage running through the bending section, and
wherein the imaging unit is disposed at an end of an imaging tool inserted into the tool channel.

6. A continuum robot control method for a continuum robot control system including a continuum robot and a control apparatus configured to control operation of the continuum robot, the continuum robot including a bending section configured to be bent with respect to a reference axis by a linear member being driven, a driving unit configured to drive the linear member, and an imaging unit located near an end of the bending section, the continuum robot control method comprising:
angle estimation where the control apparatus, in a case where a characteristic region related to a path of a lumen of a subject greater than or equal to a predetermined area is included in a field of view of the imaging unit in bending the bending section in a predetermined direction, estimates an angle restriction value for a bending angle of the bending section based on an end position of the bending section detected after insertion of the bending section into the lumen and structural information about the lumen; and
angle restriction where the control apparatus, in bending the bending section in the predetermined direction, restricts driving of the driving unit so that the bending section bends within a range of the angle restriction value.

7. A non-transitory recording medium recording a program for causing a computer to perform the continuum robot control method according to claim 6.
